(19) United States
(12) Patent Application Publication
(10) Pub. No.: US 2010/0081892 A1
(43) Pub. Date: Apr. 1, 2010

(54) Systems and Methods for Combined Pulse Oximetry and Blood Pressure Measurement

(75) Inventors: Rakesh Sethi, Vancouver (CA); Paul Stanley Addison, Edinburgh (GB); James Nicholas Watson, Dunfermline (GB)

Correspondence Address: Nellcor Puritan Bennett LLC;ATTN: IP Legal, 6135 Gunbarrel Avenue, Boulder, CO, 80301, US

(73) Assignee: NelIcor Puritan Bennett Ireland, Galway (IE)

(21) Appl. No.: 12/242,446

(22) Filed: Sep. 30, 2008

(51) Int. Cl. A61B 5/1455; A61B 5/0205

(52) U.S. Cl. 600/301; 600/344; 600/324

(57) ABSTRACT

The present disclosure relates to pulse oximetry measurements and, more particularly, relates to a combined sensor that includes a pulse oximetry (SpO₂) sensor component and a continuous non-invasive blood pressure (CNIBP) sensor component. The combined sensor can be positioned such that the SpO₂ sensor component is located over tissues where pulsatility is weak while the CNIBP sensor component may be located over tissues where pulsatility is strong. A second separate CNIBP sensor may be used to together with the CNIBP sensor component of the combined sensor in order to detect the differential pressure pulse transit time from the heart to two different locations on the body. A pulse signal detected by the CNIBP sensor component of the combined sensor can be used to trigger the SpO₂ measurement from the SpO₂ sensor component in order to improve SpO₂ measurement fidelity.

### SUMMARY

The present disclosure relates to pulse oximetry measurements and, more particularly, relates to a combined sensor that includes a pulse oximetry (SpO₂) sensor component and a continuous non-invasive blood pressure (CNIBP) sensor component.

In an embodiment, a combined sensor that includes a support structure that is coupled to an SpO₂ sensor component and a CNIBP sensor component, is provided. The SpO₂ sensor component and the CNIBP sensor component both include at least one emitter and at least one detector. The SpO₂ sensor may be located over tissues where pulsatility is weak while the CNIBP sensor component may be located over tissues where pulsatility is strong. In some embodiments, the combined sensor may be positioned on the head of a subject such that that the SpO₂ sensor component is located approximately over the subject's eyebrow while the CNIBP sensor component is located approximately over the subject's temple. A second separate CNIBP sensor may be used together with the CNIBP sensor component of the combined sensor in order to detect the differential pressure pulse transit time from the heart to two different locations on the body. A pulse signal detected by the CNIBP sensor component of the combined sensor may be used to trigger the SpO₂ measurement from the SpO₂ sensor component in order to improve SpO₂ measurement fidelity.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows a perspective view of an illustrative pulse oximetry system in accordance with an embodiment;

FIG. 2 is a block diagram of the illustrative pulse oximetry system of FIG. 1 coupled to a patient in accordance with an embodiment;

FIG. 3 is a block diagram of an illustrative signal processing system in accordance with some embodiments;

FIG. 4 shows an illustrative combined sensor that includes a pulse oximetry (SpO₂) sensor component and a continuous non-invasive blood pressure (CNIBP) sensor component in accordance with some embodiments;

FIG. 5 shows an illustrative cross-section of a combined sensor that includes a SpO₂ sensor component, a CNIBP sensor component, and a support structure in accordance with some embodiments;

FIG. 6 shows illustrative signals detected by the CNIBP and SpO₂ sensors in accordance with some embodiments;

FIG. 7 shows another illustrative combined sensor that includes a SpO₂ sensor component and a CNIBP sensor component in accordance with some embodiments; and

FIG. 8 shows an illustrative diagram of a combined sensor that may be attached to an ear in accordance with some embodiments.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.

1. First, the natural logarithm of (1) is taken (“log” will be used to represent the natural logarithm) for IR and Red

log I=log Iₒ−(sβₒ+(1−s)βᵣ)l (2)

2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{\frac{d log I ( {λ}_{R} )}{d t}}{\frac{d log I ( {λ}_{IR} )}{d t}} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{\frac{d I}{d t}}{I} \qquad ( 6 )$$

now (5) becomes

$$\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {π}_{R} )} \\ = R \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=Rx(t) (8)

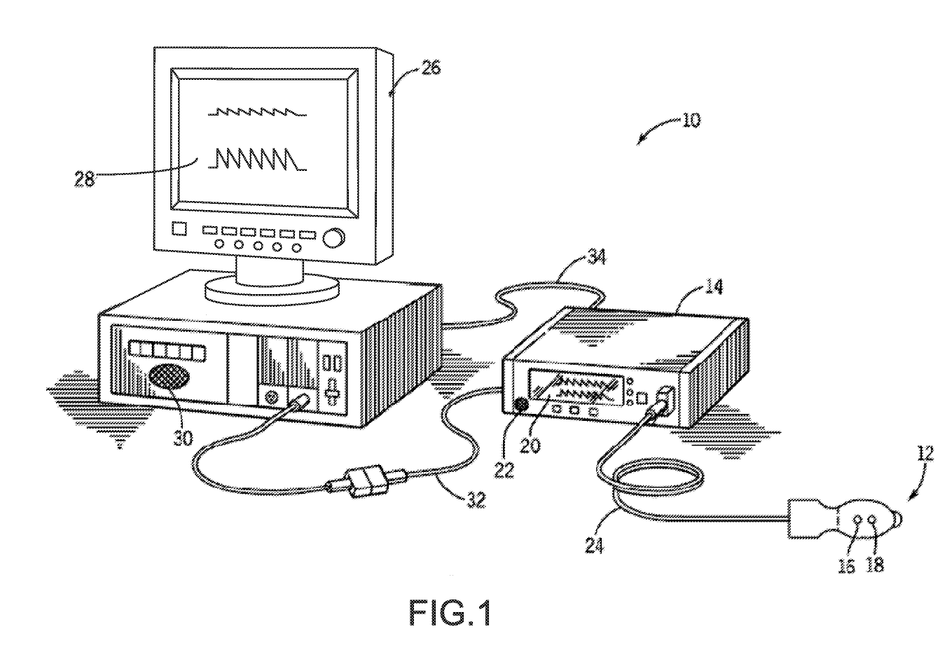

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to another embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In another embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from a blood pressure monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown), Monitor 14 may be powered by a battery (not shown) or by a conventional power source such as a wall outlet.

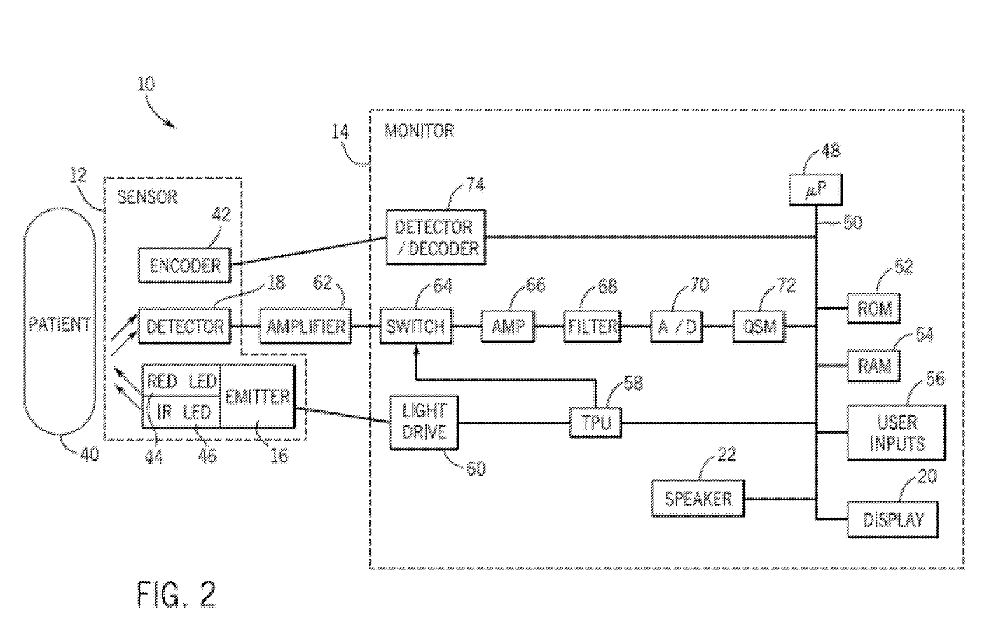

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42, In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the RED wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

Various approaches have been used for monitoring the blood pressure of living subjects. One approach is to insert a pressure sensor directly into a suitable artery of the subject. The sensor may be connected to a suitable monitoring device by a lead which passes through the subject's skin. This approach may provide highly accurate and instantaneous blood pressure measurements, but is very invasive. A surgical procedure is generally required to introduce the pressure sensor, and the fistula through which the lead exits the subject's body can provide a pathway for infection.

Another approach to measuring blood pressure uses a sphygmomanometer. A typical sphygmomanometer has an occluding cuff capable of being wrapped around a subject's arm. A pump is used to inflate the cuff, and an aneroid or mercury gravity sphygmomanometer is used to measure the pressure in the cuff, Such devices are widely used in hospitals, but are not well adapted for providing continuous blood pressure monitoring.

Some continuous non-invasive blood pressure monitoring (CNIBP) techniques have been developed that involve the use of two probes or sensors positioned at two different locations on a subject's body. The elapsed time, T, between the arrival of corresponding points of a pulse signal at the two locations may then be determined using the two probes or sensors. The estimated blood pressure, p, may then be related to the elapsed time, T, by

p=a+b·ln(T) (9)

where a and b are constants that are dependent upon the nature of the subject and the signal detecting devices. Other blood pressure equations using elapsed time may also be used. These techniques may be referred to as differential pulse transit time (DPTT) based CNIBP.

In some embodiments, the constants a and b in equation (9) may be determined by performing a calibration. The calibration may involve taking a reference blood pressure reading to obtain a reference blood pressure P₀, measuring the elapsed time T₀ corresponding to the reference blood pressure, and then determining values for both of the constants a and b from the reference blood pressure and elapsed time measurement. Calibration may be performed at any suitable time (e.g., once initially after monitoring begins) or on any suitable schedule (e.g., a periodic or event-driven schedule).

The calibration may include performing calculations mathematically equivalent to

$$\begin{matrix}a = {c}_{1} + \frac{{c}_{2} ( {P}_{0} - {c}_{1} )}{ln ( {T}_{0} ) + {c}_{2}} \\ and & ( 10 ) \\ b = \frac{{P}_{0} - {c}_{1}}{ln ( {T}_{0} ) + {c}_{2}} & ( 11 )\end{matrix}$$

to obtain values for the constants a and b, where c₁ and c₂ are predetermined constants.

In other embodiments, determining the plurality of constant parameters in the multi-parameter equation (1) may include performing calculations mathematically equivalent to

a=P₀−(c₃T₀+c₄)ln(T₀) (12)

and

b=c₃T₀+c₄ (13)

where a and b are first and second parameters and c₃ and c₄ are predetermined constants.

In some embodiments, the multi-parameter equation (9) includes a non-linear function which is monotonically decreasing and concave upward in a manner specified by the constant parameters.

Continuous and non-invasive blood pressure monitoring using these techniques is described in Chen et al. U.S. Pat. No. 6,566,251, which is hereby incorporated by reference herein in its entirety. The technique described by Chen et al. may use two sensors (e.g., ultrasound or photoelectric pulse wave sensors) positioned at any two locations on a subject's body where pulse signals are readily detected. For example, sensors may be positioned on an earlobe and a finger, an earlobe and a toe, or a finger and a toe of a patient's body.

The use of multiple probes or sensors in non-invasive continuous blood pressure monitoring provides reliable results. However, in some instances, the use of multiple separate probes or sensors at different locations on the subject's body may be cumbersome, especially for a mobile subject. Moreover, one of the multiple probes or sensors may become detached from the subject, resulting in a disruption in the continuous monitoring of the patient's blood pressure. Accordingly, some techniques for continuously monitoring a subject's blood pressure use only a single probe or sensor. In some embodiments, the single probe or sensor may detect a photoplethysmograph (PPG) signal generated, for example, by a pulse oximeter. The PPG signal may then be analyzed and used to compute a time difference between two or more characteristic points in the PPG signal. From this time difference, reliable and accurate blood pressure measurements may be computed on a continuous or periodic basis. This technique is described in more detail in U.S. patent application Ser. No. ______ (Attorney Docket No. H-RM-01205 (COV-11)), filed Sep. 30, 2008, entitled “SYSTEMS AND METHODS FOR NON-INVASIVE BLOOD PRESSURE MONITORING,” which is incorporated by reference herein in its entirety. In some embodiments, blood pressure measurements may be determined based on pulses in a PPG signal detected by a single sensor, for example, by measuring the area under a pulse or a portion of the pulse in the PPG signal. This technique is described in more detail in U.S. patent application Ser. No. ______ (Attorney Docket No. H-RM-01206 (COV-13)), filed Sep. 30, 2008, entitled “SYSTEMS AND METHODS FOR NON-INVASIVE CONTINUOUS BLOOD PRESSURE DETERMINATION,” which is incorporated by reference herein in its entirety.

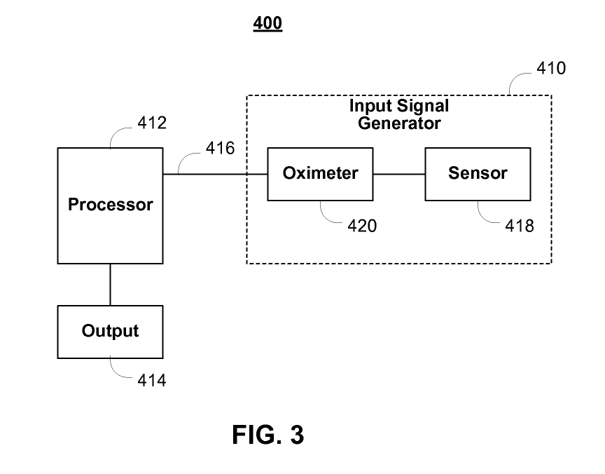

FIG. 3 is an illustrative signal processing system in accordance with an embodiment. In this embodiment, input signal generator 410 generates an input signal 416. As illustrated, input signal generator 410 may include oximeter 420 coupled to sensor 418, which may provide as input signal 416, a PPG signal. It will be understood that input signal generator 410 may include any suitable signal source, signal generating data, signal generating equipment, or any combination thereof to produce signal 416. Signal 416 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 416 may be coupled to processor 412. Processor 412 may be any suitable software, firmware, and/or hardware, and/or combinations thereof for processing signal 416. For example, processor 412 may include one or more hardware processors (e.g., integrated circuits), one or more software modules, computer-readable media such as memory, firmware, or any combination thereof. Processor 412 may, for example, be a computer or may be one or more chips (i.e., integrated circuits). Processor 412 may perform the calculations associated with the signal processing of the present disclosure as well as the calculations associated with any suitable interrogations of the transforms. Processor 412 may perform any suitable signal processing of signal 416 to filter signal 416, such as any suitable band-pass filtering, adaptive filtering, closed-loop filtering, and/or any other suitable filtering, and/or any combination thereof.

Processor 412 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 412 to, for example, store data corresponding to signal 416.

Processor 412 may be coupled to output 414. Output 414 may be any suitable output device such as, for example, one or more medical devices (e.g., a medical monitor that displays various physiological parameters, a medical alarm, or any other suitable medical device that either displays physiological parameters or uses the output of processor 412 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 400 may be incorporated into system 10 (FIGS. 1 and 2) in which, for example, input signal generator 410 may be implemented as parts of sensor 12 and monitor 14 and processor 412 may be implemented as part of monitor 14.

The present disclosure relates to a combined sensor that includes a SpO₂ sensor component and a CNIBP sensor component. Generally speaking, the location requirements for optimal detection of SpO₂ and DPTT based CNIBP may be different. As described above, CNIBP sensors can detect the differential pressure pulse transit time from the heart to two different locations on the body. These CNIBP sensors may be located over tissues where pulsatility is strong over a wide variety of perfusion conditions. Major arteries are therefore typically good sites for these CNIBP sensors. For example, typical sites for CNIBP sensors are the radial artery on the forearm and the temporal artery on the head. In contrast, typical sites that are good for measuring SpO₂ are highly perfused tissues without the presence of large pulsating absorbers such as major arteries. For example, typically sites for measuring SpO₂ are a fingertip, toe, forehead or earlobe.

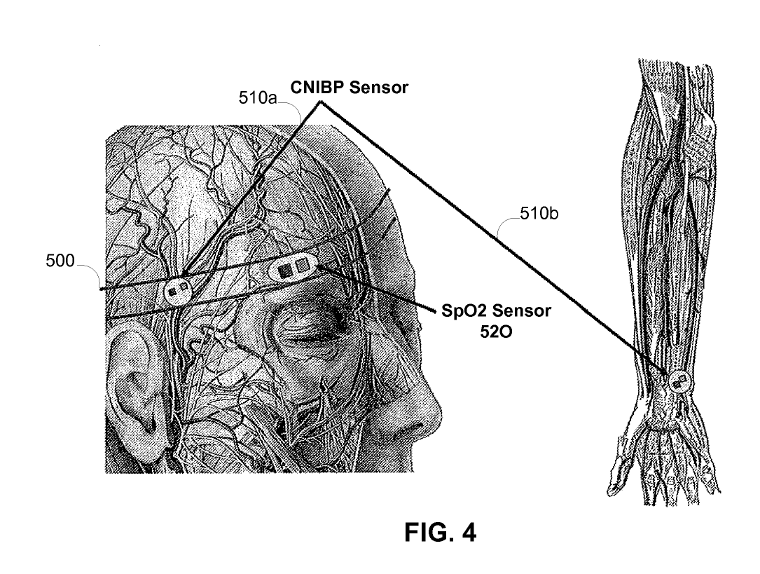

FIG. 4 shows an illustrative sensor 500 containing a first CNIBP sensor component 510a positioned approximately over the temporal artery and SpO₂ sensor component 520 positioned approximately over the eyebrow. The area around the temporal artery is a strong pulsatility site which may be suitable for CNIBP measurement. The area around the eyebrow, in contrast, has low pulsatility which may be suitable for SpO₂ measurement. A second CNIBP sensor 510b may be positioned over the radial artery on the wrist. As described above, DPTT based CNIBP techniques may use two sensors positioned at two different locations on a subject's body to estimate blood pressure by measuring an amount of time between the arrival of corresponding points of a pulse signal at the two locations. In some embodiments, single sensor CNIBP monitoring techniques, such as those described above, may be used. Using these techniques, only first CNIBP sensor component 510a may be required for measuring blood pressure. In one single sensor CNIBP monitoring technique, an amount of time between two or more characteristic points of a pulse signal detected by the single sensor may be measured. In another single sensor CNIBP monitoring technique, an area under one or more portions of a pulse signal detected by the single sensor may be measured. In some embodiments, both sensor components may be used to measure CNIBP and SpO₂ signals at both sites, then one of the signals may be selected or the two signals may be combined.

CNIBP sensor components 510a and 510b may include a single wavelength emitter and detector for detecting pulsatility of the arteries. The emitter detector separation and wavelength selection of CNIBP sensors 510a and 510b may be optimized for detecting pulsatility. For example, the wavelength of the emitter and detector of the of CNIBP sensors 510a and 510b may be an IR wavelength.

SpO₂ sensor component 520 may measure oxygen saturation using, for example, using the ratio of ratios technique described above or any other suitable technique. SpO₂ sensor component 520 may include a dual wavelength emitter and a detector for measuring the absorption of light in the tissue. For example, SpO₂ sensor component 520 may include dual emitters for red and IR wavelengths. The emitter detector separation and wavelength selection of SpO₂ sensor component 520 may be optimized for measuring the intensity of light that is received at the sensor as a function of time.

Using the ratio of ratios SpO₂ measurement technique, the intensities of two wavelengths detected by SpO₂ sensor component 520 at different points in the pulse cycle may be compared to measure oxygen saturation levels. The upstroke portion of the detected PPG signal may provide the best results for using this measurement technique. When SpO₂ sensor component 520 is in a relatively low perfusion site, such as around the eyebrow, it may be difficult to directly detect the upstroke portion of the PPG signal when, for example, noise or artifacts are present in the signal. In contrast, CNIBP sensor components 510a and 510b, located over major arteries, may more easily detect the location of upstrokes in the PPG signal. Thus, the pulse signal detected by one or both CNIBP sensor components 510a and 510b may be used to trigger the ratio of ratios calculation for SpO₂ measurement. For example finding the period of upstroke of the pressure pulse may involve taking the first derivative of the CNIBP signal and using the portions with a sustained value above a trigger threshold to identify suitable, upstroke, time periods. Using a pulse signal detected by CNIBP sensor components 510a and 510b to trigger the ratio of ratios calculation for SpO₂ measurement in this manner may improve the SpO₂ measurement fidelity and may minimize the affect of noise and artifacts.

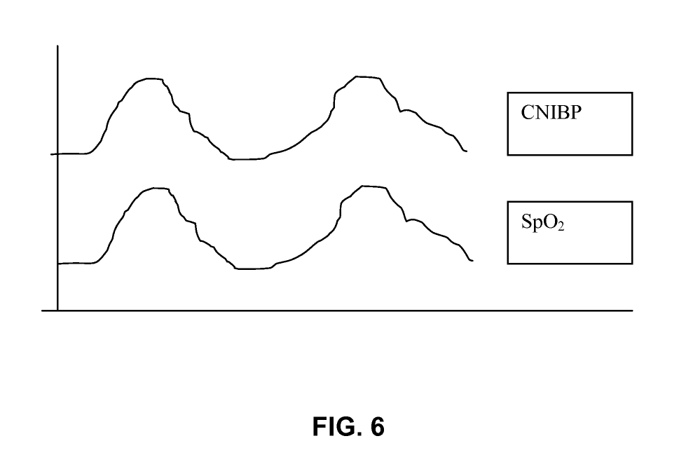

Typically the signals detected by the CNIBP and SpO₂ sensors are similar. FIG. 6 shows illustrative signals detected by the CNIBP and SpO₂ sensors. In an embodiment, the CNIBP and SpO₂ signals both include at least one transmission or reflection signal received from an optical emitter of common wavelength, for example both may use an IR emitting source. The main difference in signal morphology may be caused by the different site locations (e.g., one capillary and one arterial) which may make the CNIBP signal more pulsatile, with increased high frequency components, and less affected by noise. A second difference between the two signals may be the sampling frequency. For example, the CNIBP sensor may sample at a much faster rate (e.g., 1 KHz or every 1 millisecond) that the SpO₂ sensor (e.g., 75 Hz or every 13.3 milliseconds). The time difference between head sites (forehead/ear) from the finger is approximately 60 milliseconds, though it does vary from individual to individual. Therefore, if a CNIBP sensor were being used by the SpO₂ system (for example to identify characteristic points of the pleth or artifact) the pulse arrival times from finger to head may differ by approximately 4 samples which may be considered irrelevant when detecting a pulse for SpO₂ calculation. However, where the CNIBP and SpO₂ sensors are proximal it is reasonable to assume that pulses may be observed at the same time by the two detectors.

As a processor (e.g., processor 412 (FIG. 4)) receives both signals it may, in one embodiment, use the CNIBP signal to improve the accuracy of a SpO2 calculation through its application to the selection of useful data. In an alternative embodiment the CNIBP signal may be used to trigger a measurement from the SpO2 sensor for use in the derivation of a saturation value, for example, during the upstroke of a pulse.

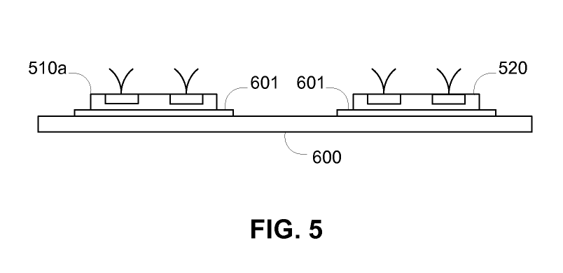

FIG. 5 shows an illustrative cross-section of sensor 500 containing a first CNIBP sensor component 510a, SpO₂ sensor component 520, and support structure 600 coupled to both sensors. Sensor components 510a and 520 may be secured to support structure 600 using adhesive 601 or any other suitable attachment technique. Further, while adhesive 601 is shown as securing the underside of sensor components 510a and 520 to support structure 600, it should be understood that sensor components 510a and 520 may be secured over support structure 600 (as shown), under support structure 600, or at least partially embedded in support structure 600. Similarly, adhesive 601 or an equivalent attachment medium may be located under the sensor components (as shown), around the sensor components, over the sensor components, or some combination thereof. In some embodiments one or more of the sensor components may be integrated with or built directly onto support structure 600. Support structure 600 may be made of any suitable material or combination of materials. Support structure 600 may be made of a flexible material that allows sensor components 510a and 520 to achieve close contact with desired sensor site locations, even when those locations are across a curved surface such as a patient's head. As discussed above, positioning the two sensor components in close proximity may reduce delays between the sensors. However, it should be noted that where CNIBP and SpO₂ sensors are close the issue of crosstalk must be minimized or compensated for.

Sensor 500 may be attached to a patient using any suitable approach. For example, as shown in FIG. 4, sensor 500 may be attached to a patients head using a headband. Such a headband may be directly attached to sensor components 510a and 520 as a support structure or may be attached to a separate support structure 600. In some embodiments, sensor 500 may attach directly to a patient using, for example, an integrated adhesive area or using any other suitable approach. Alternatively sprung clips may be used to measure capillary sites used for SpO₂ (e.g., ear lobe or fingertip) while adhesive sensors may be more suited for the arterial sites used for CNIBP measurements.

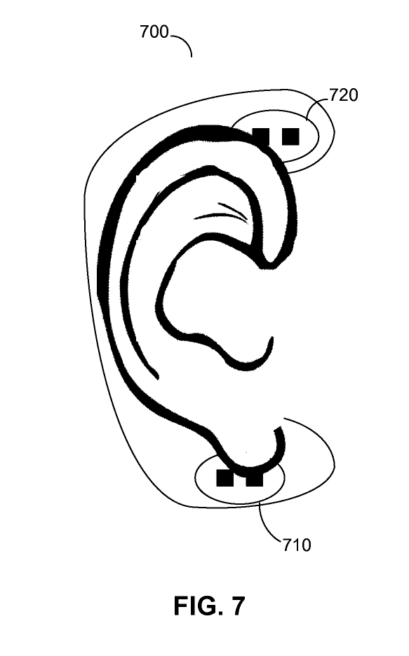

Another site that may be used for the combined sensor includes locations around and on the ear. FIG. 7 shows an illustrative sensor 700 containing a CNIBP sensor component 710 and an SpO₂ sensor component 720. CNIBP sensor component 710 may be positioned around the bottom of the ear, underneath the earlobe on the side of the face and neck. This sensor location exhibits strong pulsatility and may be a good site for measurement of strong pulsations suitable for CNIBP measurements. SpO₂ sensor component 720 positioned around the side of the face at the top of the ear over hard bone behind the “helix.” Alternatively SpO₂ sensor component 720 may also be positioned on the ear lobe itself (not shown).

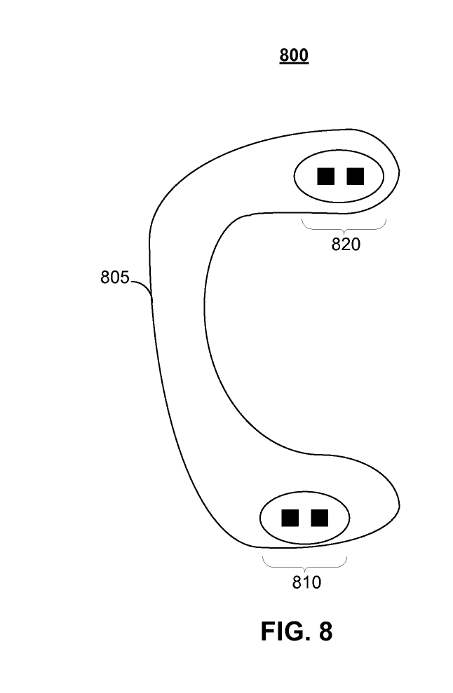

FIG. 8 shows an illustrative diagram of a combined sensor 800 that may be attached to an ear in the same manner as sensor 700 (FIG. 7). Sensor 800 includes deformable foam support structure 805 which may be used to attach two sensor components 810 and 820, one optimized for SpO₂ and one optimized for CNIBP, around the ear. The foam support structure 805 may have adhesive at each end, at the sensor component sites and may be deformable in the middle part to allow the sensor 800 to be bent around the ear. In some embodiments, support structure 805 can loops around the ear to provide additional support.

It will be understood that sensor 500 or 700 may be used in place of sensor 12 in system 10 (FIGS. 1 and 2) or in place of input signal generator 410 in system 400 (FIG. 3)

The foregoing is merely illustrative of the principles of this disclosure and various modifications can be made by those skilled in the art without departing from the scope and spirit of the disclosure. The following claims may also describe various aspects of this disclosure.

What is claimed is:

1. A sensor, comprising:
a support structure;
a pulse oximetry (SpO₂) sensor component comprising at least one emitter and at least one detector, wherein the SpO₂ sensor component is coupled to the support structure; and
a continuous non-invasive blood pressure (CNIPB) sensor component comprising at least one emitter and at least one detector, wherein the CNIBP sensor component is coupled to the support structure.

2. The sensor of claim 1, wherein the support structure is a flexible support structure.

3. The sensor of claim 1, wherein the support structure is capable of being attached to a subject such that the SpO₂ sensor component is positioned over tissue having weak pulsatility and such that the CNIPB sensor component is simultaneously positioned over tissue having strong pulsatility.

4. The sensor of claim 1, wherein the support structure is capable of being attached to a head of a subject such that the SpO₂ sensor component is positioned approximately over an eyebrow of the subject and such that the CNIPB sensor component is simultaneously positioned approximately over a temporal artery of the subject.

5. The sensor of claim 1, wherein the support structure is capable of being attached to a head of a subject such that the SpO₂ sensor component is positioned on the head of the subject near a top of an ear of the subject and such that the CNIPB sensor component is simultaneously positioned on the head of the subject underneath an earlobe of the subject.

6. The sensor of claim 1, wherein the CNIBP sensor component coupled to the support structure is a first CNIBP sensor component, the sensor further comprising a second CNIBP sensor component that is not coupled to the support structure.

7. The sensor of claim 1, wherein the CNIBP sensor component is a single wavelength sensor component.

8. The sensor of claim 1, wherein the SpO₂ sensor component is a dual wavelength sensor component.

9. A pulse oximetry and blood pressure monitor, comprising:
a combined sensor comprising:
a support structure;
a pulse oximetry (SpO₂) sensor component comprising at least one emitter and at least one detector, wherein the SpO₂ sensor component is coupled to the support structure;
a continuous non-invasive blood pressure (CNIPB) sensor component comprising at least one emitter and at least one detector, wherein the CNIBP sensor component is coupled to the support structure; and
a processor capable of measuring pulse oximetry and blood pressure based at least in part on a pulse signal detected by CNIBP sensor component and a photoplethysmograph (PPG) signal detected by the SpO₂ sensor component.

10. The monitor of claim 9, wherein the combined sensor support structure is a flexible support structure.

11. The monitor of claim 9, wherein the combined sensor support structure is capable of being attached to a subject such that the SpO₂ sensor component is positioned over tissue having weak pulsatility and such that the CNIPB sensor component is simultaneously positioned over tissue having strong pulsatility.

12. The monitor of claim 9, wherein the combined sensor support structure is capable of being attached to a head of a subject such that the SpO₂ sensor component is positioned approximately over an eyebrow of the subject and such that the CNIPB sensor component is simultaneously positioned approximately over a temporal artery of the subject.

13. The monitor of claim 9, wherein the combined sensor support structure is capable of being attached to a head of a subject such that the SpO₂ sensor component is positioned on the head of the subject near a top of an ear of the subject and such that the CNIPB sensor component is simultaneously positioned on the head of the subject underneath an earlobe of the subject

14. The monitor of claim 9, wherein the CNIBP sensor component coupled to the support structure is a first CNIBP sensor component, the monitor further comprising a second CNIBP sensor component that is not coupled to the support structure.

15. The monitor of claim 14, wherein the processor is capable of measuring blood pressure based at least in part on a calculated differential pulse transit time (DPTT) between a portion of a pulse signal detected by the first and the second CNIBP sensor components.

16. The monitor of claim 9, wherein the processor is capable of measuring blood pressure based at least in part on a calculated amount of time between two or more portions of a pulse signal detected by the CNIBP sensor component.

17. The monitor of claim 9, wherein the processor is capable of measuring blood pressure based at least in part on a calculated amount of area underneath a portion of a pulse signal detected by the CNIBP sensor component.

18. The monitor of claim 9, wherein the processor is capable of measuring pulse oximetry levels using a photoplethysmograph (PPG) signal detected by the SpO₂ sensor component.

19. The monitor of claim 18, wherein the measuring of pulse oximetry levels relies, at least in part, on the signal detected by the CNIBP sensor component.

20. A method for measuring blood oxygen saturation and blood pressure, comprising:
detecting a photoplethysmograph (PPG) signal with a pulse oximetry (SpO₂) sensor component of a combined sensor comprising at least one emitter and at least one detector;
detecting a pulse signal with a continuous non-invasive blood pressure (CNIPB) sensor component of the combined sensor comprising at least one emitter and at least one detector; and
measuring pulse oximetry and blood pressure based at least in part on the detected PPG signal and the detected pulse signal.

21. The method of claim 20, further comprising positioning the SpO₂ sensor component over tissue having weak pulsatility and simultaneously positioning the CNIPB sensor component over tissue having strong pulsatility.

22. The method of claim 20, further comprising positioning the SpO₂ sensor component over an eyebrow of a subject and simultaneously positioning the CNIPB sensor component over a temporal artery of the subject.

23. The method of claim 20, further comprising positioning the SpO₂ sensor component near a top of an ear of a subject and simultaneously positioning the CNIPB sensor component underneath an earlobe of the subject.

24. The method of claim 20, wherein measuring blood pressure comprises calculating a differential pulse transit time (DPTT) between a portion of a pulse signal detected by the CNIBP sensor component and a second CNIBP sensor component.

25. The method of claim 20, wherein measuring blood pressure comprises calculating an amount of time between two or more portions of a pulse signal detected by the CNIBP sensor component.

26. The method of claim 20, wherein measuring blood pressure comprises calculating an area underneath a portion of a pulse signal detected by the CNIBP sensor component.

27. The method of claim 20, wherein measuring pulse oximetry levels comprises using a photoplethysmograph (PPG) signal detected by the SpO₂ sensor component.

28. The method of claim 20, wherein measuring of pulse oximetry levels relies, at least in part, on the signal detected by the CNIBP sensor component.
